(19) United States
(12) Patent Application Publication
(10) Pub. No.: US 2014/0194833 A1
(43) Pub. Date: Jul. 10, 2014

(54) ACCESS PORT

(71) Applicant: HeartWare, Inc., Miami Lakes, FL (US)

(72) Inventor: Lance Lynn Andrus, SOUTHBOROUGH, MA (US)

(73) Assignee: HEARTWARE, INC., Miami Lakes, FL (US)

(21) Appl. No.: 14/147,321

(22) Filed: Jan. 3, 2014

Related U.S. Application Data
(60) Provisional application No. 61/749,068, filed on Jan. 4, 2013.

(51) Int. Cl. A61B 17/02

(52) U.S. Cl. CPC A61B 17/0218
USPC 604/288.02

(57) ABSTRACT

An access port includes a first and a second resilient element, each having a first face and a second face. A first slit and a second slit are defined in the first and second resilient elements, respectively. The first face of the first resilient element abuts the first face of the second resilient element. The lengthwise direction of the second slit is transverse to the lengthwise direction of the first slit. The first and second resilient elements are configured to assume at least a first position wherein the first and second slits are substantially closed, thereby preventing a passage of a fluid therebetween and at least a second position wherein a member projects through the first and second slits, thereby allowing the member to pass through the access port.

### CROSS REFERENCE TO RELATED APPLICATIONS

This application claims the benefit of U.S. Provisional Application No. 61/749,068, filed Jan. 4, 2013, the disclosure of which is hereby incorporated by reference in its entirety.

### FIELD OF INVENTION

The present invention relates to an access port and more particularly to an access port for allowing access of a member through the port while providing a fluid seal.

### BACKGROUND OF THE INVENTION

It is often necessary to provide access through an access port which generally provides at least a low pressure fluid seal. For example, a tool may be required to project through the access port occasionally or frequently. It may be desirable to have an access port which provides such a fluid seal while allowing a tool to project therethrough with a minimal fluid leakage through the access port. In some cases, it is desirable for the access port to provide a fluid seal once the tool is removed. For example, in certain medical applications a tool such as a hypodermic needle, catheter or other elongated body may project through the seal during certain stages of a medical procedure, and the tool may be removed during other stages of the procedure.

### BRIEF SUMMARY OF THE INVENTION

An embodiment of the invention includes an access port comprising first and second resilient elements. The first resilient element has a first face and a second face and comprises a first slit extending through the element from the first face to the second face. The first slit is elongated, and extends in a lengthwise direction along the first and second faces. The second resilient element has a first face and a second face and comprises a second slit extending through the element from the first face to the second face. The second slit is elongated, and extends in a lengthwise direction along the first and second faces. The first face of the second resilient element abuts the first face of the first resilient element. The lengthwise direction of the second slit is transverse to the lengthwise direction of the first slit. The first and second resilient elements are configured to assume at least a first position wherein the first and second slits are substantially closed, thereby preventing a passage of a fluid therebetween. The first and second resilient elements are configured to assume at least a second position wherein a member extends through the first and second slits.

According to an aspect of the invention, the access port further comprises a third resilient element having a first face and a second face and comprising a third slit extending through the element from the first face to the second face. The first face of the third resilient element abuts the second face of the second resilient element. The third slit has a lengthwise direction transverse to the lengthwise direction of the second slit. In an embodiment of the invention, the lengthwise direction of the third slit is transverse to the lengthwise directions of the first and second slits. In an embodiment of the invention, the first, second and third slits are defined in the first, second, and third elements without removing any material from the first, second and third resilient elements.

In an embodiment of the invention, the second slit is oriented substantially perpendicular to the first slit. In another embodiment of the invention, the lengthwise direction of the second slit is oriented at about 60° with the lengthwise direction of the first slit and the lengthwise direction of the third slit is oriented at about 120° with the lengthwise direction of the first slit.

According to an embodiment of the invention, the first and second resilient elements comprise silicone. In an exemplary embodiment, the access port further comprises a lubricant between the first face of the first resilient element and the first face of the second resilient element and between the second face of the second resilient element and the first face of the third resilient element.

### BRIEF DESCRIPTION OF THE DRAWINGS

Various embodiments of the present invention will now be discussed with reference to the appended drawings. It is to be appreciated that these drawings depict only some embodiments of the invention and are therefore not to be considered limiting of its scope.

FIG. 1 is an exploded view of an access port, according to an embodiment of the invention;

FIG. 2 is a perspective view of the access port of FIG. 1;

FIG. 3 is an end view of a member projecting through the access port of FIGS. 1 and 2;

FIG. 4 is a perspective view of an access port, according to another embodiment of the invention;

FIGS. 5 is an end view of a member projecting through the access port of FIG. 4;

FIG. 6A is a cross-sectional view of the access port of FIG. 1 arranged in a catheter and in a closed position, prior to a member projecting through it, according to an embodiment of the invention; and

FIG. 6B is a cross-sectional view of the catheter of FIG. 6A after the member has projected the access port.

### DETAILED DESCRIPTION

The following discussion describes, in detail, various aspects and embodiments of the present invention. This discussion should not be construed as limiting the invention to those particular aspects or embodiments. Rather, practitioners skilled in the art will recognize numerous other aspects and embodiments as well, which are within the scope of the present invention.

In describing the embodiments of the present invention illustrated in the drawings, specific terminology will be used for the sake of clarity. For purposes of explanation, the invention is generally described herein with regard to an access port. However, the present invention is not intended to be limited to the specific terms so selected

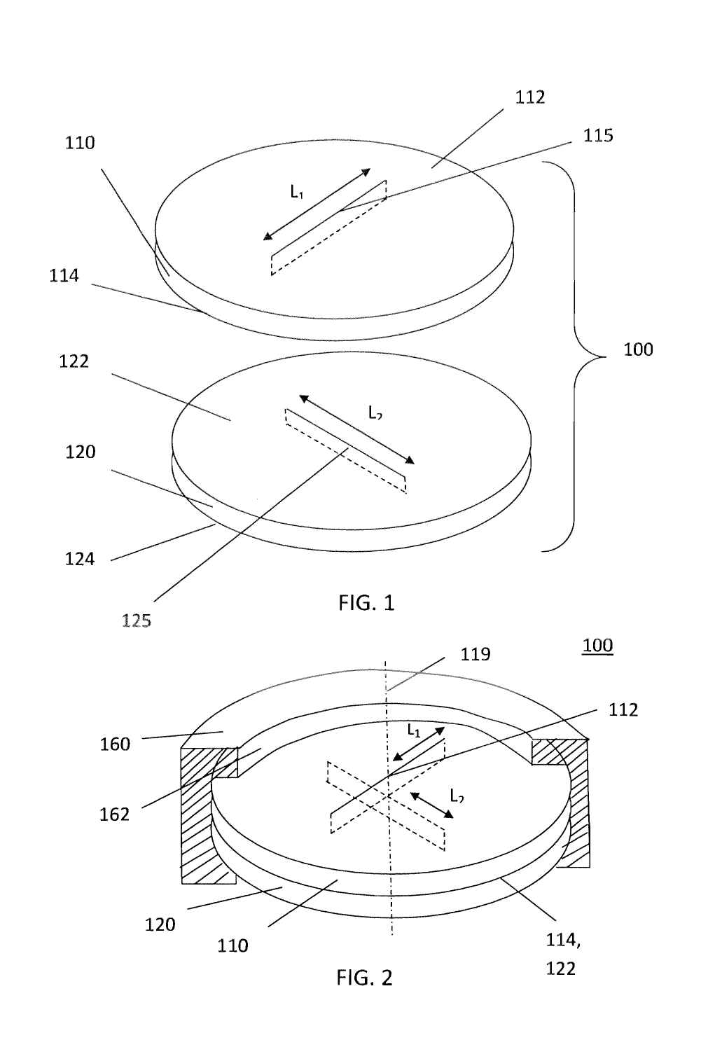

Referring now to FIG. 1, an access port 100 is illustrated in an exploded view. The access port 100 comprises a stack of a first resilient member 110 and a second resilient member 120. The first resilient member 110 comprises a first face 112 and a second face 114. Likewise, the second resilient member 120 comprises a first face 122 and a second face 124. In an exemplary embodiment, the first member 110 and second member 120 may take the form of a generally planar disc. As best seen in FIG. 2, the second member 120 abuts the first member 110 such that the faces 122, 114 are in contact engagement with one another. In this embodiment, the first and second resilient elements 110, 120 are compressed against one another by a housing 160 which includes a ring surrounding the resilient elements 110, 120 and a pair of lips 162 bearing on the first surface 112 of the first member and on the second surface 124 of the second member. Only a portion of the housing 160 is depicted in FIG. 2 with other portions removed for clarity of illustration. The housing 160 may be formed as a single, unitary part, or may have multiple parts. Also, the housing 160 may include features other than the ring.

The first resilient member 110 comprises a first slit 115 defined therein. The first slit 115 extends through first member 110, from the first surface 112 to the second surface 114. Also, the first slit extends in a first lengthwise direction L₁ along surfaces 112 and 114. Similarly, the second resilient member 120 comprises a second slit 125 defined therein. The second slit 125 extends through the second member 120, from first face 122 to second face 124, and extends in a second lengthwise direction L₂. The first slit 115 and the second slit 125 desirably are cut in the first resilient member 110 and the second resilient member 120, respectively, such that material of the members 110, 120 is simply displaced and not removed. In their initial or natural configurations the slits 115, 125 tend to remain in a closed position so as to prevent passage of a fluid therethrough.

The first and second members 110, 120 are oriented so that the slits 115, 125 cross one another at a crossing axis 119 extending in the direction through the members 110, 120, normal to the surfaces of the members 110, 120.

In the illustrated embodiment, the crossing axis 119 lies at a center point of the first resilient member 110 and a center point of the second resilient member 120, respectively. For instance, in a case of generally circular first and second resilient members 110, 120, as depicted in FIGS. 1 and 2, the first and second slits 115, 125 extend lengthwise along a diameter of the first resilient member 110 and a diameter of the second resilient member 120, respectively. In an exemplary embodiment, the length of the first slit 115 and the second slit 125 may be in the range of about 50% to about 60% of the respective diameters of the first and second resilient members 110, 120. Of course, in other embodiments, the lengths of the first and second slits 115, 125 may be different, depending on the requirements of a given application.

The first slit 115 and the second slit 125 are oriented transverse to one another. In the embodiment of FIGS. 1 and 2, the first and second lengthwise directions L₁ and L₂ are generally perpendicular to one another. In other embodiments, the first and second lengthwise directions L₁ and L₂ may be oriented at a different angle, ranging from greater than 0° to about 90°.

In an exemplary embodiment, the first resilient member 110 and the second resilient member 120 may be take the form of elastomeric discs. By way of a non-limiting example, the first resilient member 110 and the second resilient member 120 may be made of a rubber such as silicone rubber, natural or synthetic polyisoprene rubber or butyl rubber. Other resilient materials such as solid or foamed polymeric materials with an appropriate elasticity to function as described herein also can be used. The dimensions, proportions and physical properties of the resilient elements 110, 120 will vary with the size and intended application of the access port 100. In one exemplary embodiment, if the access port 100 comprises of a stack of first and second resilient elements about 50 mm in diameter and intended for use in a medical device to provide access for a member such as a hypodermic needle, catheter or trocar of about 20 mm diameter, the elastomer used may have a durometer ranging from about 40 to about 60 and have a thickness ranging from about 0.25 mm (or inches) to about 0.75 mm (or inches).

In an exemplary embodiment, the first resilient element 110 and the second resilient element 120 may be identical to one another in shape, size, material, durometer, and thickness. In other embodiments, the first resilient element 110 and the second resilient element 120 may be differ from one another in one or more of these characteristics.

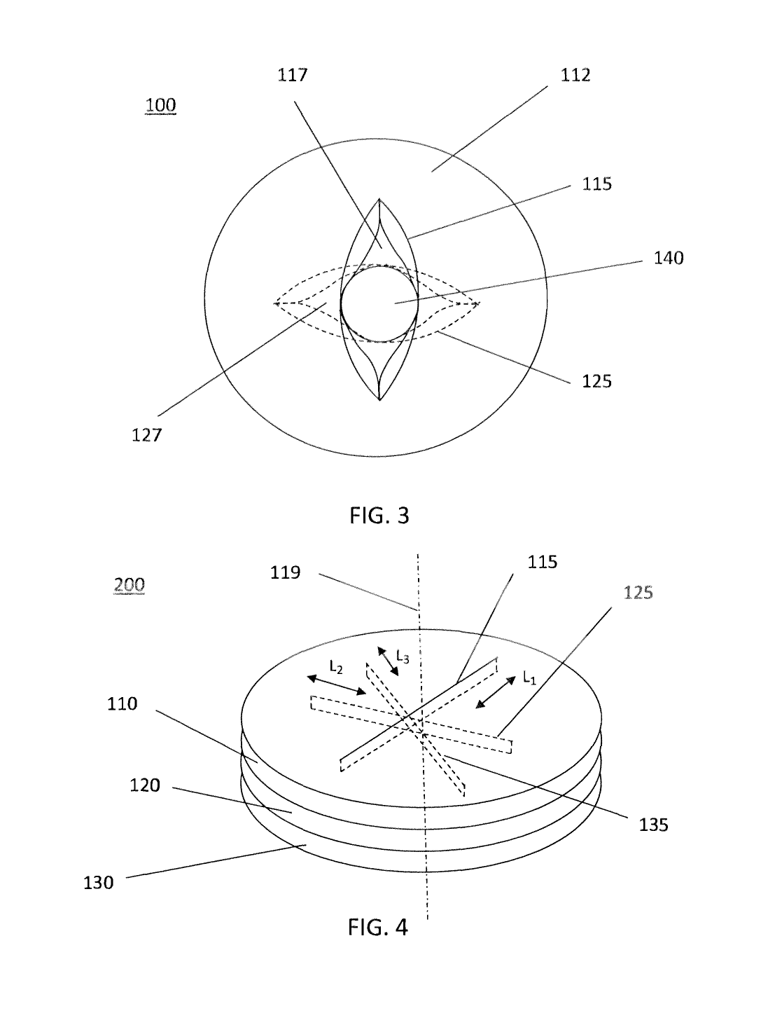

Referring now to FIG. 3, a schematic illustration depicts a member 140 projecting through the first slit 115 and the second slit 125. Although FIG. 3 illustrates a generally circular member 140, it will be understood that members of other geometrical cross-sectional shapes may also be accommodated by the access port 100. In an exemplary embodiment, the first slit 115 and the second slit 125 may accommodate a member 140 having a diameter up to about 75% of the lengths of the first slit 115 and the second slit 125. Of course, in other embodiments, the member 140 of a different diameter may be accommodated by the access port 100 by using appropriate values of one or more of the durometer and the thickness of one or more of the first resilient element 110 and the second resilient element 120. Once the member 140 is withdrawn from the first slit 115 and the second slit 125, the first resilient element 110 and the second resilient element 120 snap back or revert to their natural closed positions, thereby substantially preventing passage of any fluid through the slits 115, 125 and creating at least a low-pressure fluid seal.

Although the present invention is not limited by any theory of operation, it is believed that when the member 140 projects through slit 115, openings or leak paths 117 may be created in the first slit 115 in the first resilient element 110, at regions remote from the crossing axis 119. However, the second resilient element 120, which abuts the first resilient element 110, substantially covers the openings or leak paths 117, thereby minimizing leakage of a fluid through the access port. Likewise, openings or leak paths 127 in the second resilient element remote from the crossing axis 119 are substantially blocked by the abutting first resilient element 110. It is believed that this blocking action minimizes leakage while the member 140 is in place. Likewise, when the member 140 is removed, some leak paths may be present due to imperfect resilience of members 110 and 120. Here again, however, it is believed that the abutting first member substantially blocks any such leakage paths in the second member, and vice-versa.

Referring now to FIG. 4, an access port 200 is illustrated according to another embodiment of the invention. The access port 200 comprises a stack of the first resilient element 110, the second resilient element 120, and a third resilient element 130. The third resilient element 130 abuts the second resilient element 120 in a fashion similar to that of the second resilient element 120 abutting the first resilient element 110. The third resilient element 130 comprises a third slit 135. Here again, all of the slits cross one another at a crossing axis 119. The second lengthwise direction L₂ of second slit 125 is oriented transverse to the first lengthwise direction L₁ of first slit 115, and the third lengthwise direction L₃ third slit 135 is oriented transverse to the second lengthwise direction L₂ of second slit 125. In an exemplary embodiment, the second lengthwise direction of the second slit 125 may be oriented at an angle of about 60° to the first lengthwise direction of first slit 115 and the third lengthwise direction of the third slit 135 may be oriented at an angle of about 120° to the first lengthwise direction of the first slit 115. In other embodiments, the angles of orientation of the second slit 125 relative to the first slit 115 and of the third slit 135 relative to the first slit 115 may be varied, according to the requirements of a given application. For example, the first and third lengthwise directions L₁ and L₃ of the slits in the first and third elements may be parallel to one another, whereas the second lengthwise direction L₂ of the slit in the second element may be perpendicular to the first and third lengthwise directions L₁ and L₃. Even in this arrangement, however, the lengthwise direction of the slit in each element is transverse to the lengthwise direction of the slit in each immediately adjacent, abutting element.

It will be understood that in other configurations the stack may include more than three resilient elements, each having a slit transverse to the slit in the abutting resilient element. In these embodiments as well, the resilient elements 110, 120, 130 may be compressed against one another by a housing (not shown), for example a ring surrounding the resilient elements 110, 120, 130.

The resilient members in an access port having three or more such members may be thinner or softer than the members of a comparable access port having only two resilient members.

The access ports discussed above optionally may include a lubricant (not shown) at each interface between abutting surfaces of the resilient elements. By way of non-limiting example only, the lubricant may be a silicone lubricant, silicon oil or water based lubricant. An advantage of using a lubricant is the resilient elements remain substantially unconstrained by one another, so that the resilience of the elements may more readily close the slits upon removal of the member. Another advantage of using a lubricant is that a formation of natural bonding between some grades of elastomers of the resilient elements may be prevented or minimized.

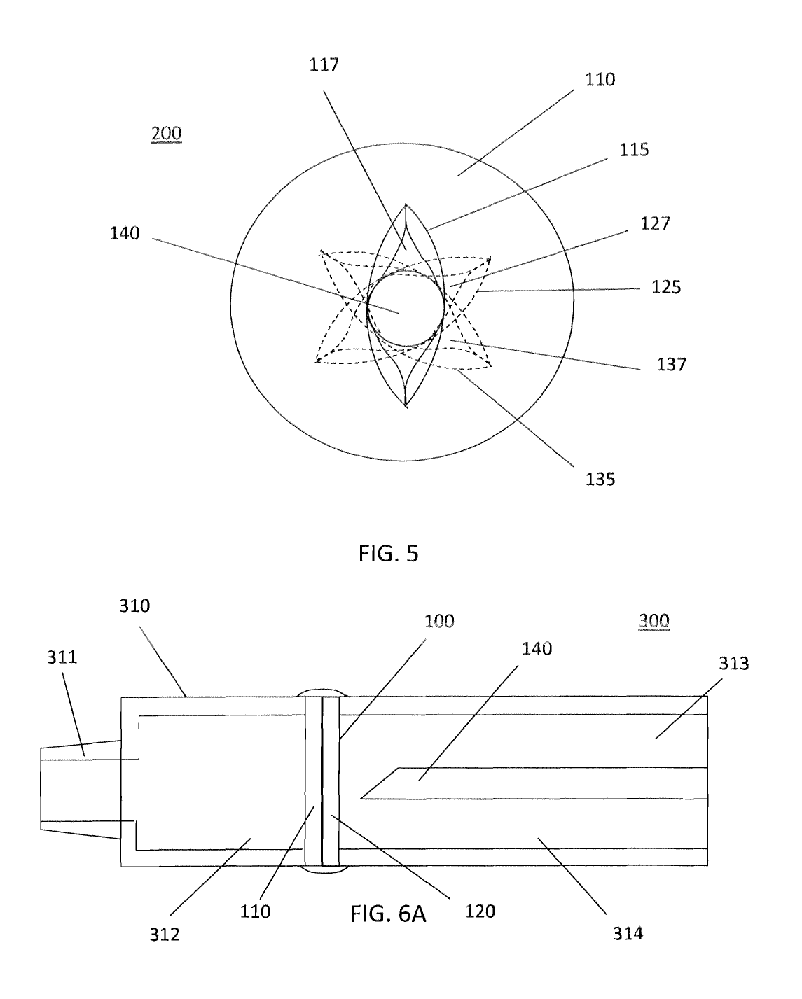

FIG. 5 illustrates an end view of the access port 200. A member 140 is shown projecting through the first slit 115, the second slit 125, and the third slit 135. When a member 140 projects through the slits 115, 125, 135 of the access port 200, leak paths or openings 117, 127, 137 are respectively created in the first, second and third slits 115, 125, 135. However, the opening 117 is substantially covered by the second resilient member 120. Likewise, the opening 127 is substantially covered by the third resilient member 130. Thus, it will be understood that with addition of more adjacent and abutting resilient elements, each of the opening or leak path created by projection of a member 140 will be covered by the adjacent resilient member, thereby minimizing or substantially preventing leakage of a fluid through the access port.

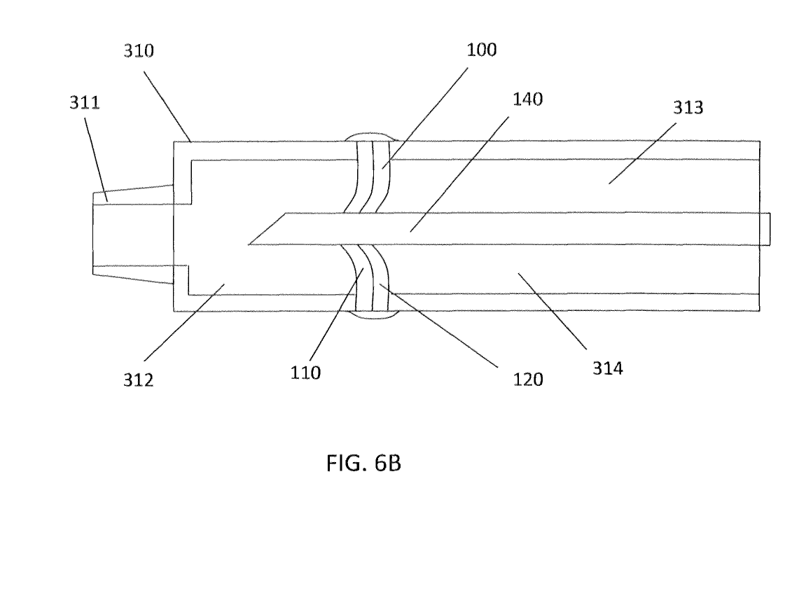

Referring now to FIGS. 6A and 6B, an exemplary application of the access port 100 is schematically illustrated. The access port 100 is arranged in the hub 310 of a catheter 311. The catheter 311 projects from a closed end of the hub, whereas the opposite end 313 is open. In this embodiment, the hub 310 of the catheter serves as the housing for the access port 100, and holds the resilient elements 110 and 120 in abutting relationship with one another. In the natural or closed configurations, the slits of the access port members 110, 120 are closed and provide at least a low-pressure seal for a fluid between a first side 312, at the closed end of the hub and a second side 314 at the open end of the hub. FIG. 6A illustrates a state wherein a member 140 such as a hypodermic needle is not projecting through the access port 100. FIG. 6B, on the other hand, illustrates a state wherein the member 140 projects through the slits of the access port 100. Because of the transverse orientation of the abutting resilient elements 110, 120, the leak path or opening created in each slit by the passage of the member 140 is substantially covered by the abutting resilient member, thereby minimizing leakage of a fluid. When the needle or member 140 is withdrawn from the access port 100, the resilient elements 110, 120 revert to their natural closed configuration, thereby re-establishing fluid seal between the sides 312 and 314 of the catheter hub 310. Merely by way of example, the seal provided by the access port can be used to block leakage of blood out through the open end 313 of the hub during insertion of the catheter into the body of a patient and upon removal of the needle.

It will be appreciated that the access ports 100 and 200 are not limited to only catheters and can be utilized wherever a through access of a member is desired while maintaining a fluid seal while providing access through the access port. For example, the access ports can be made in larger sizes to accommodate trocars or other medical devices. In still further embodiments, the access ports can be used in non-medical applications. Merely by way of example, access ports according to embodiments of the present invention can be used to provide access for electrical cables or mechanical components through a bulkhead or wall.

In the embodiments discussed above, each of the abutting surfaces is planar. However, this is not essential. In other embodiments, the abutting surfaces may take other forms, for example, a generally convex surface of one member may abut a generally concave surface of the immediately adjacent member. Also, it is not essential for the surfaces of adjacent members to abut one another over the entire extent of the surfaces. However, the surfaces of adjacent members desirably abut one another at the slots and in regions adjacent the slots.

An advantage of the slits described above is that for every element of the stack, a leak path is substantially covered by the adjacent element. Thus, in a stack comprising a plurality of resilient elements with each slit oriented transverse to the slit of the preceding element, the leak paths become smaller and smaller, when a member is projected through the slits of the access port. Thus, given a sufficient number of resilient elements in a stack, much higher pressure fluid seal may be achieved.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. An access port comprising:
a first resilient element having a first face and a second face, and comprising a first slit defined therein, the first slit extending through the first resilient element from the first face to the second face, and extending in a first lengthwise direction;
a second resilient element having a first face and a second face, the second resilient element comprising a second slit defined therein, the second slit extending through the second resilient element from the first face of the second resilient element to the second face of the second resilient element, and extending in a second lengthwise direction;
wherein the first face of the second resilient element abuts the first face of the first resilient element;
wherein the second lengthwise direction is transverse to the first lengthwise direction and wherein the first and second slits cross one another at a crossing axis; and
wherein the first and second resilient elements are configured to assume at least a first position wherein the first and second slits are substantially closed, thereby preventing a passage of a fluid therebetween; and
wherein the first and second resilient elements are configured to assume at least a second position wherein a member projects through the first and second slits, thereby allowing the member to pass through the access port.

2. The access port of claim 1, wherein the first and second resilient elements comprise an elastomer.

3. The access port of claim 2, wherein the elastomer comprises silicone.

4. The access port of claim 1, wherein the first element and the second element comprise first and second discs, respectively.

5. The access port of claim 4, wherein the first slit passes through a center point of the first disc and the second slit passes through a center point of the second disc.

6. The access port of claim 5, wherein the first slit extends about 50% to about 60% of a diameter of the first resilient element.

7. The access port of claim 5, wherein the second slit extends about 50% to about 60% of a diameter of the second resilient element.

8. The access port of claim 4, wherein a diameter of the first resilient element is equal to a diameter of the second resilient element.

9. The access port of claim 1, wherein the second slit is substantially perpendicular to the first slit.

10. The access port of claim 1, further comprising a lubricant between the first face of the first resilient element and the first face of the second resilient element.

11. The access port of claim 10, wherein the lubricant comprises a silicone lubricant.

12. The access port of claim 1, further comprising a third resilient element having a first face and a second face, and comprising a third slit defined therein, the third slit extending through the third resilient element from the first face of the third resilient element to the second face of the third resilient element, and extending in a third lengthwise direction;
wherein the first face of third resilient element abuts the second face of the second resilient element; and
wherein the third lengthwise direction is transverse to the second lengthwise direction and wherein the third slit crosses the first and second slits at the crossing axis.

13. The access port of claim 12, wherein the third lengthwise direction of the third slit is transverse to the first lengthwise direction of the first slit.

14. The access port of claim 12, wherein the second lengthwise direction is oriented at an angle of about 60° with the first lengthwise direction; and
wherein the third lengthwise direction is oriented at an angle of about 120° with the first lengthwise direction.

15. The access port of claim 12, wherein the first, second, and third resilient elements comprise a first, a second, and a third disc, respectively.

16. The access port of claim 12, wherein the first, second, and the third resilient elements are compressed against one another.

17. The access port of claim 12, further comprising:
a lubricant between the first face of the first resilient element and the first face of the second resilient element; and
a lubricant between the second face of the second resilient element and the first face of the third resilient element.

18. The access port of claim 1, wherein the first and second slits are defined in the first and second resilient elements without removing any material from the first and second elements.

19. The access port of claim 1, wherein the first and second resilient elements are compressed against one another.
